(19) United States
(12) Patent Application Publication
(10) Pub. No.: US 2021/0382067 A1
(43) Pub. Date: Dec. 9, 2021

(54) HIGH-THROUGHPUT AND MASS-SPECTROMETRY-BASED METHOD FOR QUANTITATING ANTIBODIES

(71) Applicant: Regeneron Pharmaceuticals, Inc., Tarrytown, NY (US)

(72) Inventors: Yuetian YAN, Chappaqua, NY (US); Shunhai WANG, Scarsdale, NY (US)

(21) Appl. No.: 17/341,746

(22) Filed: Jun. 8, 2021

Related U.S. Application Data
(60) Provisional application No. 63/036,679, filed on Jun. 9, 2020.

(51) Int. Cl. G01N 33/68

(52) U.S. Cl. CPC G01N 33/6857

(57) ABSTRACT

Liquid chromatography-free methods for quantitating a target protein in a sample are provided. One embodiment provides a liquid chromatography-free method for quantifying target antibodies in a sample including the steps of spiking the sample with a labeled internal standard antibody, digesting the antibodies in the sample to produce peptides, fractionating the peptides; and quantifying the target antibodies using a direct infusion MS² system containing one or more ion traps and two or more quadrupole mass filters and an electrospray ionizer, wherein the method is liquid chromatography-free

### CROSS-REFERENCE TO RELATED APPLICATIONS

This application claims benefit of and priority to U.S. Provisional Patent Application No. 63/036,679 filed on Jun. 9, 2020, which is incorporated by reference in its entirety.

### TECHNICAL FIELD OF THE INVENTION

This invention is generally related to systems and methods for quantitating antibodies.

### REFERENCE TO SEQUENCE LISTING

The instant application contains a Sequence Listing which has been submitted electronically in ASCII format and is hereby incorporated by reference in its entirety. Said ASCII copy, created on Jun. 2, 2021, is named 064752_032US1_SL.txt and is 601 bytes in size.

### BACKGROUND OF THE INVENTION

For the development of antibody-based therapeutics, reliable quantitation of the drug molecules in animal serum/plasma samples is critical to support toxicokinetic and pharmacokinetic studies. Liquid chromatography coupled to tandem mass spectrometry (LC-MS/MS) methods have been increasingly applied to quantitate therapeutic peptides and proteins in complex biological matrixes, due to its advantages in method development time, specificity, selectivity, feasibility of multiplexing, and wide dynamic range, comparing to ligand binding assays (LBAs). However, conventional LC-MS-based assays often suffer from low throughput, for example only 100 samples can be processed per day using LC-MS.

Therefore, it is an object of the invention to provide more efficient and sensitive systems and methods for quantitating human monoclonal antibodies (mAbs) in a sample.

It is another object of the invention to provide systems and methods that can quantify protein concentration in more than 100 samples per day.

### SUMMARY OF THE INVENTION

Liquid chromatography-free methods for quantitating a target protein in a sample are provided. One embodiment provides a liquid chromatography-free method for quantifying target antibodies in a sample including the steps of spiking the sample with a labeled internal standard antibody, digesting the antibodies in the sample to produce peptides, fractionating the peptides; and quantifying the target antibodies using a direct infusion MS² system containing one or more ion traps and two or more quadrupole mass filters and an electrospray ionizer, wherein the method is liquid chromatography-free. In some embodiments, the method further includes the step of spiking the peptides with labeled Fc peptide VVSVLTVLHQDWLNGK (SEQ ID NO:1) prior to fractionation. In one embodiment the peptides are fractionated by reverse phase solid phase extraction. The labeled internal standard antibody and the labeled Fc peptide are typically labeled with a heavy isotope. In some embodiments the heavy isotope is selected from the group consisting of ¹³C, ¹⁵N, and ²H. In one embodiment the target antibody is a human monoclonal antibody.

Still another embodiment provides a method of quantitating a protein drug product in a biological sample including the steps of spiking the sample with a known amount of a heavy isotope labeled peptide standard having an amino acid sequence according to SEQ ID NO:1, digesting protein drug product in the sample into peptides, fractionating the peptides under conditions that retain peptides having an amino acid sequence according to SEQ ID NO:1, analyzing the sample containing the protein drug product peptides and the peptide standards for the presence of the peptide having an amino acid sequence according to SEQ ID NO:1 using an MS² system to calibrate the system, wherein the MS² system comprises one or more ion traps and two or more quadrupole mass filters and an electrospray ionizer, and quantitating the amount of protein drug product present in the sample based upon the presence of the peptide, wherein the method does not utilize liquid chromatography. The protein drug product can be an antibody or antigen binding fragment thereof, a fusion protein, or a recombinant protein. In some embodiments the data for quantifying drug product ions and mass-tagged peptide standard ions are acquired in different MS² scans. As described above, the peptides are fractionated using reverse phase solid phase extraction using 15 to 25% acetonitrile as a wash and 20 to 30% acetonitrile elution. In one embodiment, a 20% acetonitrile wash and 24% acetonitrile elution is used.

In one embodiment the method further includes the step of spiking the sample of protein drug product with a heavy isotope-labeled protein drug product prior to digesting the sample.

In some embodiments the sample contains blood or serum. The blood or serum can be human or non-human. In one embodiment the serum is monkey serum.

In one embodiment the disclosed methods have a dynamic range of 1 to 1000 μm/mL and a Lower Limit of Quantification (LLOQ) of 1-2 μg/mL.

In still another embodiment the disclosed methods are automated high throughput methods.

### BRIEF DESCRIPTION OF THE DRAWINGS

FIG. 1 is a schematic illustration of the workflow of an exemplary method disclosed herein.

FIGS. 2A-2C are diagrams showing the workflow of exemplary methods disclosed herein.

FIGS. 3A-3F are exemplary graphs showing sequential parallel reaction monitoring (PRM) acquisition of endogenous and internal standard (IST) peptides.

FIGS. 4A-4C are exemplary graphs showing wide-range co-isolation of endogenous and IST peptides for PRM.

FIGS. 5A-5E are exemplary graphs showing 2-plexed PRM acquisition.

FIG. 6A is a mass spectrum graph of endogenous and spiked-in peptide y14++ acquired using wide isolation PRM at 1 μg/mL. FIG. 6B is a mass spectrum graph of endogenous and spiked-in peptide y14++ acquired using 2-plexed PRM at 1 μg/mL.

FIG. 7A is a table showing the product ions tested in FIGS. 7B-7D. FIGS. 7B-7E are mass spectra of endogenous and spiked-in y8+ and y14++ product ions in blank samples or 10 μg/mL samples of a mAb of interest.

FIG. 8A is a schematic diagram of the stepwise acetonitrile (ACN) gradient elution from an exemplary method disclosed herein. FIG. 8B is a graph showing the VVSV peptide distribution percent across an ACN stepwise gradient. FIG. 8C is a graph showing VVSV peptide intensity using different ACN elution windows (18% wash, 24% elute; 18% wash, 26% elute; 20% wash, 24% elute; 20% wash, 26% elute).

FIGS. 9A-9B are mass spectrum graphs showing relative abundance of y14++ product ion in an Oasis SPE plate washed with 18% ACN and eluted with 24% ACN (FIG. 9A) and in a Strata X-SPE plate washed with 20% ACN and eluted with 24% ACN.

FIGS. 10A-10B are calibration curves showing intensity of heavy peptide signal over various concentrations of heavy peptide. The data was fitted as a linear regression model with 1/x weighting.

FIGS. 11A-11B are calibration curves showing normalized response over various protein concentrations for samples spiked with heavy mAb internal standard. The data was fitted using a linear regression model with 1/x weighting.

FIG. 12 is a table showing QC sample analysis using the disclosed methods to detect antibody concentration.

FIGS. 13A-13B are mass spectrum graphs showing relative abundance of endogenous and SIL peptides in serum blank (FIG. 13A) and serum+internal standard mAb (FIG. 13B).

FIG. 14 is a table showing the determination of LLOQ using different lots of monkey serum.

FIG. 15A-15B are calibration curves showing relative response (FIG. 15A) and intensity (FIG. 15B) over various concentrations of mAb1 in monkey serum. FIG. 15C is a table showing the results of QC sample analysis.

FIG. 16 is a bar graph showing that increased wash volume improves LLOQ. The X-axis represents wash volume and the Y-axis represent response at 1 μg/mL mAb/blank.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

“Protein” refers to a molecule comprising two or more amino acid residues joined to each other by a peptide bond. Protein includes polypeptides and peptides and may also include modifications such as glycosylation, lipid attachment, sulfation, gamma-carboxylation of glutamic acid residues, alkylation, hydroxylation and ADP-ribosylation. Proteins can be of scientific or commercial interest, including protein-based drugs, and proteins include, among other things, enzymes, ligands, receptors, antibodies and chimeric or fusion proteins. Proteins are produced by various types of recombinant cells using well-known cell culture methods, and are generally introduced into the cell by genetic engineering techniques (e.g., such as a sequence encoding a chimeric protein, or a codon-optimized sequence, an intronless sequence, etc.) where it may reside as an episome or be integrated into the genome of the cell.

“Antibody” refers to an immunoglobulin molecule consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has a light chain variable region and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. The term antibody also includes bispecific antibody, which includes a heterotetrameric immunoglobulin that can bind to more than one different epitope. Bispecific antibodies are generally described in U.S. Pat. No. 8,586,713, which is incorporated by reference into this application.

“Fc fusion proteins” comprise part or all of two or more proteins, one of which is an Fc portion of an immunoglobulin molecule, which are not otherwise found together in nature. Preparation of fusion proteins comprising certain heterologous polypeptides fused to various portions of antibody-derived polypeptides (including the Fc domain) has been described, e.g., by Rath, T., et al., Crit Rev Biotech, 35(2): 235-254 (2015), Levin, D., et al., Trends Biotechnol, 33(1): 27-34 (2015)) “Receptor Fc fusion proteins” comprise one or more extracellular domain(s) of a receptor coupled to an Fc moiety, which in some embodiments comprises a hinge region followed by a CH2 and CH3 domain of an immunoglobulin. In some embodiments, the Fc-fusion protein comprises two or more distinct receptor chains that bind to one or more ligand(s). For example, an Fc-fusion protein is a trap, such as for example an IL-1 trap or VEGF trap.

The term “liquid chromatography-free” means that the technique of liquid chromatography is not utilized in the disclosed methods and systems.

### II. High-Throughput and Mass-Spectrometry-Based Method for Quantitating Antibodies

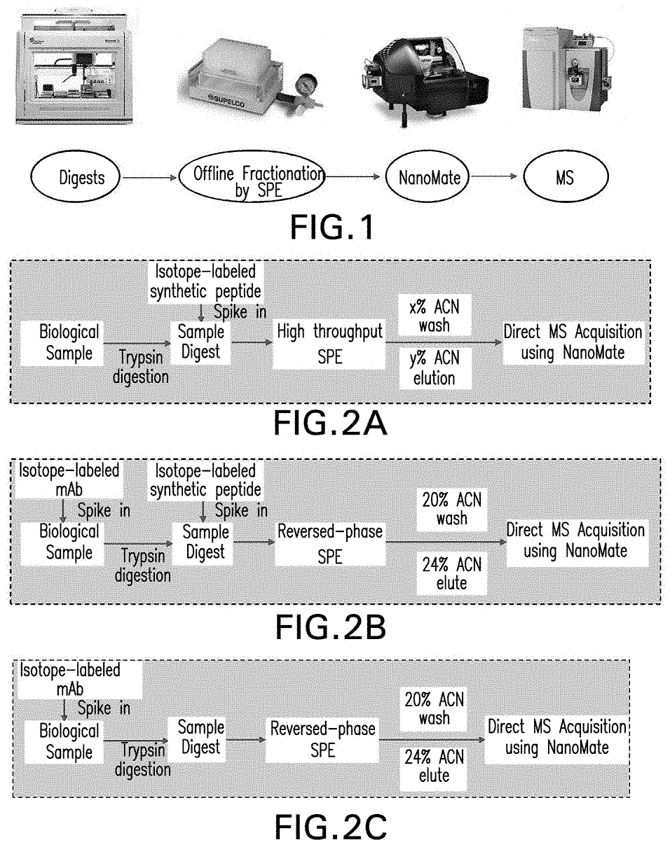

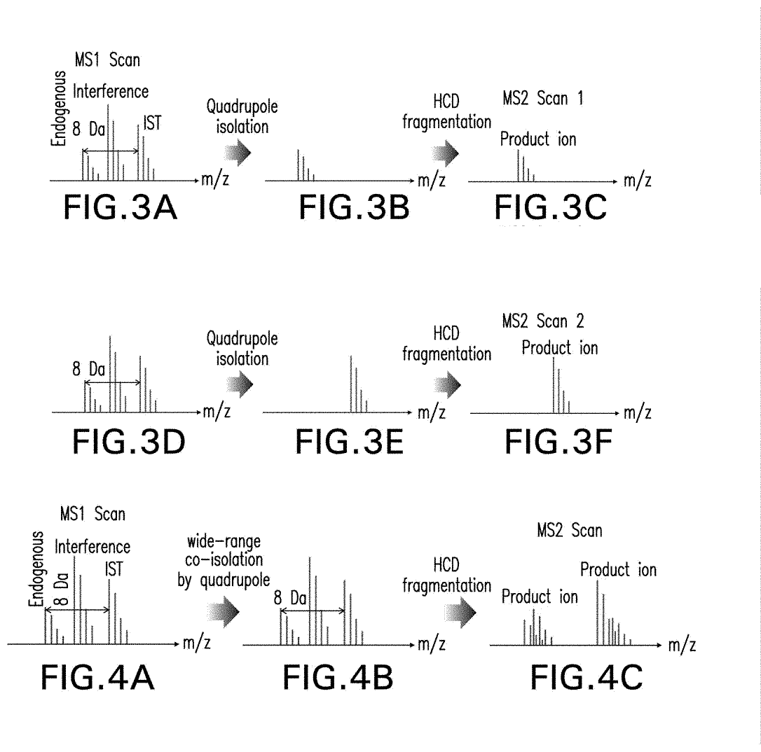

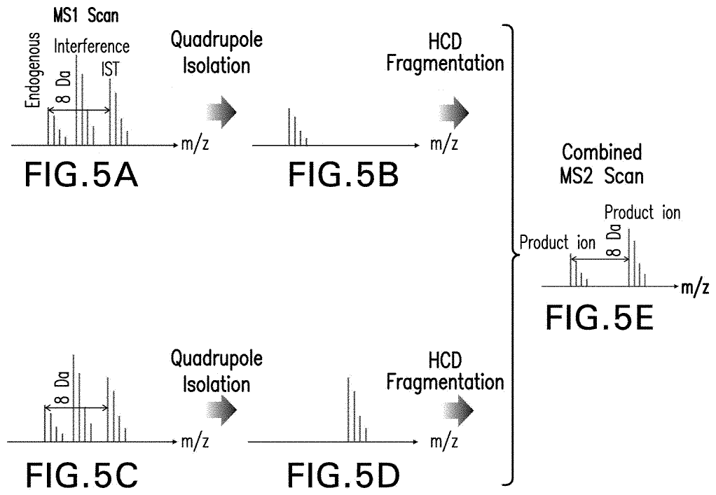

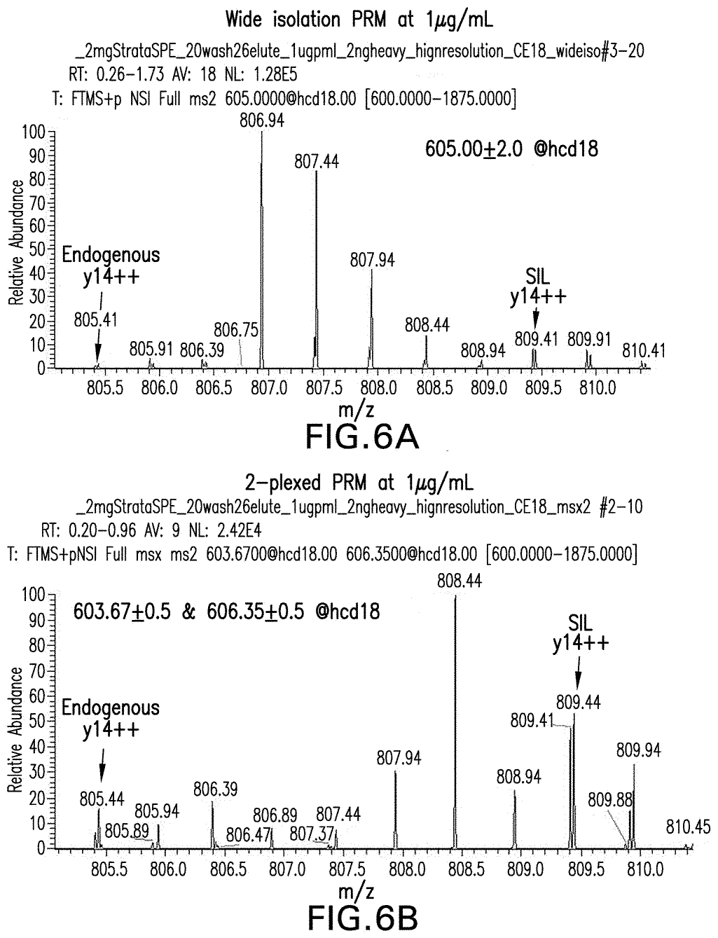

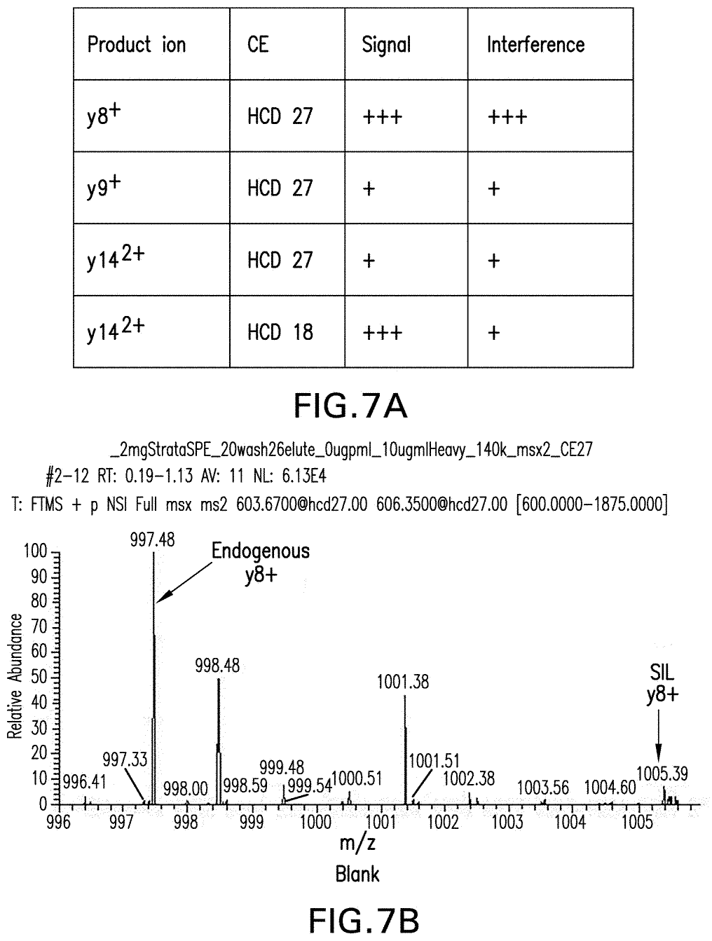

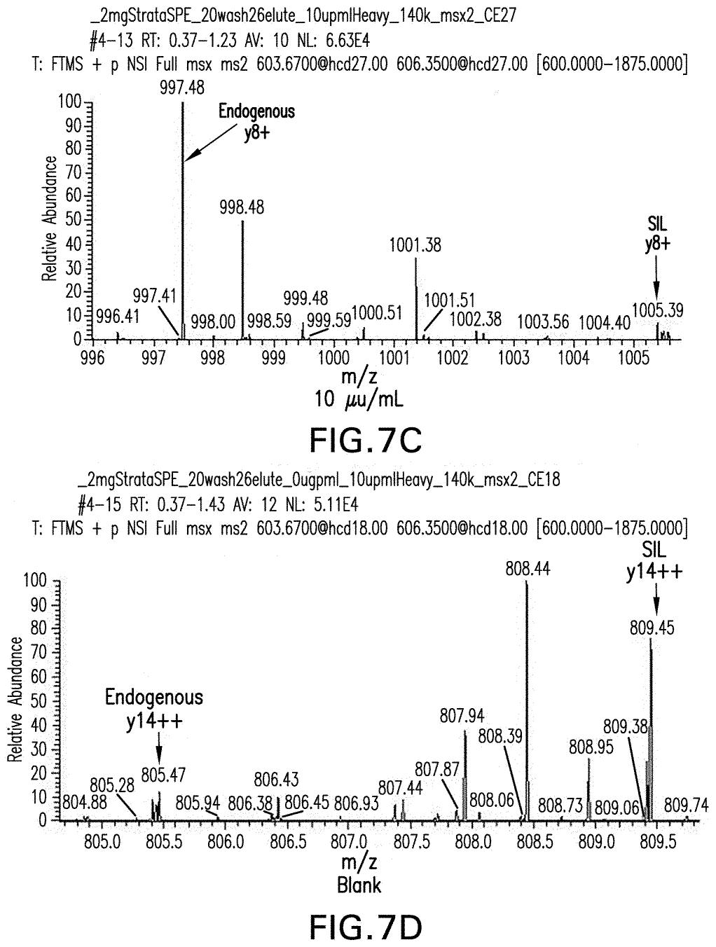

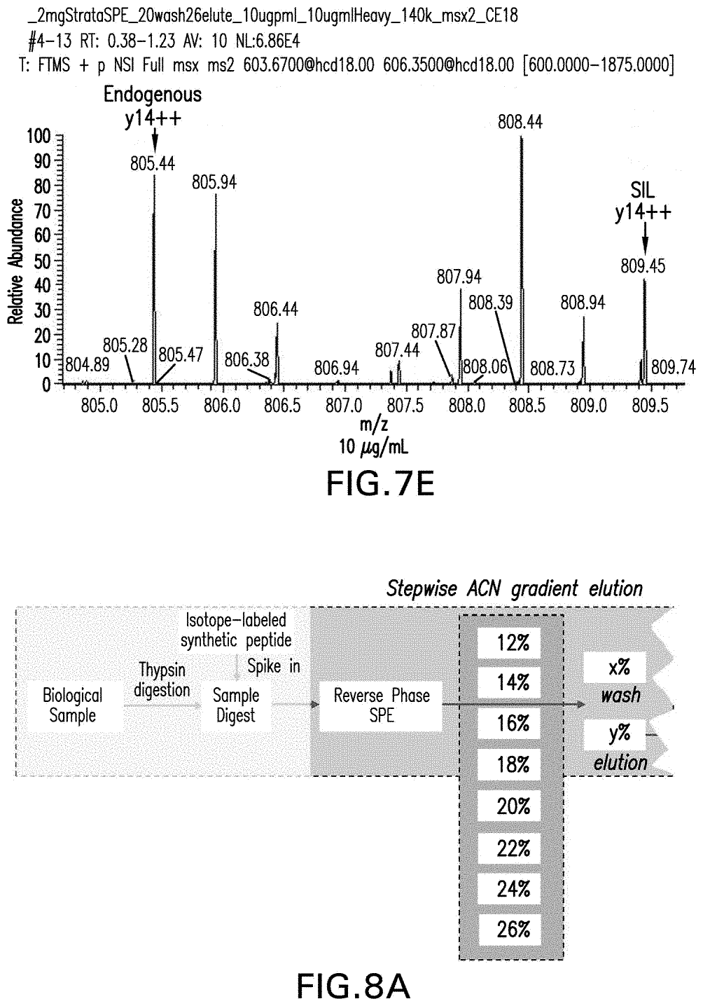

Disclosed herein are systems and methods for quantitating protein drug products in a sample, for example in non-human matrixes. In one embodiment, the protein drug product is an antibody or antigen-binding fragment thereof, a fusion protein, or a recombinant protein. The antibody is typically a monoclonal antibody. Accurate and reliable quantitation of protein drug product molecules in animal serum/plasma samples is critical to support toxicokinetic and pharmacokinetic studies during the development of protein-based and antibody-based therapeutics. Another embodiment provides high-throughput systems and methods including a liquid chromatograph-free (LC-free), parallel reaction monitoring (PRM)-based mass spectrometry (MS) method for quantitating mAbs, typically human antibodies, in a sample (FIG. 1). Another embodiment provides a method utilizing nano-spray based direct infusion for high throughput analysis (<1 min per sample, zero cross-run contamination) and a universal surrogate peptide (VVSVLTVLHQDWLNGK (SEQ ID NO:1)) from the Fc region as an internal control for total human mAb quantitation in a sample.

An exemplary liquid chromatography-free method includes digesting the protein sample into peptides, spiking in a heavy isotope labelled-peptide standard having the amino acid sequence of the surrogate peptide such as SEQ ID NO:1, fractionating the sample, and analyzing the sample using a direct infusion MS system containing one or more ion traps, two or more quadrupole mass filters, and an electrospray ionizer (FIG. 2A).

Still another embodiment provides a liquid chromatography-free method for quantifying antibody concentration in a sample including the steps of spiking the sample with an internal standard, for example a labeled antibody, digesting the antibodies in the sample to produce peptides, separating the peptides, for example using solid phase extraction, and quantifying the amount of antibody in the sample using a direct infusion MS system. In one embodiment, the direct infusion MS system includes one or more ion traps, two or more quadrupole mass filters, and an electrospray ionizer (FIG. 2B).

Yet another embodiment provides a liquid chromatography-free method for quantifying target antibodies in a sample including the steps of spiking the sample with a labeled standard antibody, digesting the antibodies in the sample to produce peptides, fractionating the peptides, and quantifying the target antibodies using a direct infusion MS system containing one or more ion traps and two or more quadrupole mass filters and an electrospray ionizer (FIG. 2C).

Further details of the methods and systems are provided in the sections below.

A. Digestion

In one embodiment, the protein or protein drug product of interest, for example an antibody or antigen-binding fragment thereof, fusion protein, or a recombinant protein, is digested into peptides typically in a 96 well plate. In one embodiment a labelled internal standard peptide, for example SEQ ID NO:1 is spiked into the sample containing target antibodies, and then the sample is subjected to protein digestion. In another embodiment, the sample containing the target antibodies is spiked with a labeled standard antibody and then subjected to digestion.

Methods of digesting proteins are known in the art. Proteins can be digested by enzymatic digestion with proteolytic enzymes or by non-enzymatic digestion with chemicals. Exemplary proteolytic enzymes for digesting proteins include but are not limited to trypsin, pepsin, chymotrypsin, thermolysin, papain, pronase, Arg-C, Asp-N, Glu-C, Lys-C, and Lys-N. Combinations of proteolytic enzymes can be used to ensure complete digestion. Exemplary chemicals for digesting proteins include but are not limited to formic acid, hydrochloric acid, acetic acid, cyanogen bromide, 2-nitro-5-thiocyanobenzoate, and hydroxylamine.

In one embodiment, the digestion step of the method is performed using 96 well plates in the Biomek® FXP Automated Workstation from Beckman Coulter which provides the speed and performance critical to today's research environments. The flexible platform is available in single and dual pipetting head models combining multichannel (96 or 384) and Span-8 pipetting, and is ideal for high throughput workflows.

In one embodiment, the sample is diluted with 8 M urea, trypsinized overnight at a ratio of 1 to 10 under reduced conditions. Exemplary reducing agents include 2-Mercaptoethanol and Dithiothreitol (DTT). In one embodiment the sample is reduced with 10 mM DTT.

B. Fractionation

After digestion, the sample is subject to fractionation to separate the digested peptides. In one embodiment, the sample is fractionated under conditions that allow for the retention of the internal standard peptide (VVSVLTVLHQDWLNGK; (SEQ ID NO:1)) and removal of the majority of other interferences for improved method sensitivity. In one embodiment, the fractionation is performed using solid phase extraction, in particular reverse phase solid phase extraction in a 96 well plate.

1. Solid Phase Extraction

Solid phase extraction (SPE) parameters were explored by comparing several commercially available SPE products including Oasis HLB reverse phase 30 mg plate, Oasis HLB reverse phase 10 mg plate, Strata-X reverse phase 10 mg plate, Strata-X reverse phase 2 mg plate, Strata-XC strong cation exchange mix mode plate, and the Strata-XA strong Anion exchange mix mode plate.

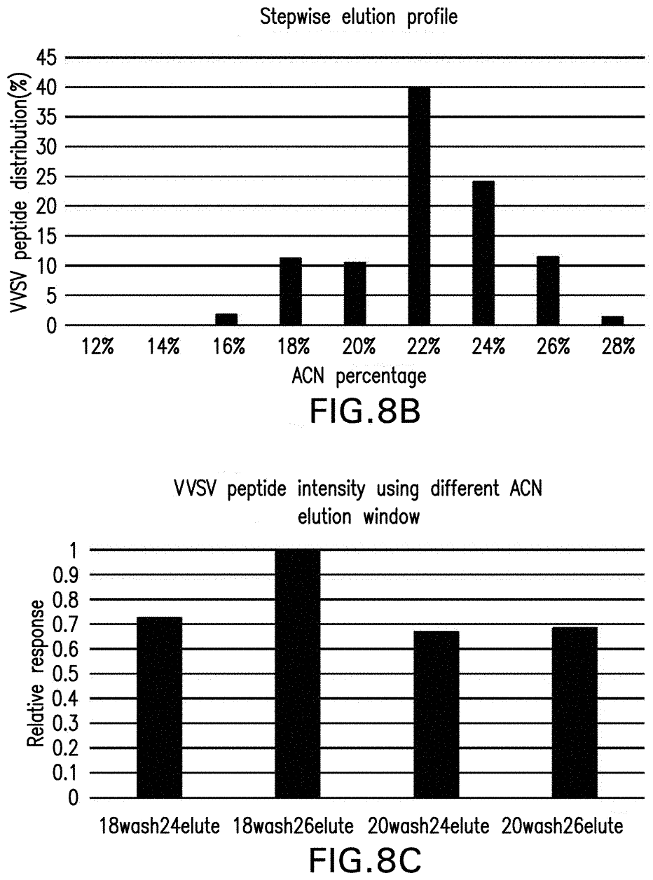

Wash and elution parameters were investigated on digested samples on the commercially available plates using 12%, 14%, 16%, 18%, 20%, 22%, 24%, 26% acetonitrile (ACN) (FIG. 8A). FIG. 8B shows the stepwise elution profile. FIG. 8C shows the internal control peptide intensity determined by mass spectrometry analysis under the indicated wash and elute concentrations of ACN. A 20% ACN wash with a 24% ACN elution was determined to be optimal.

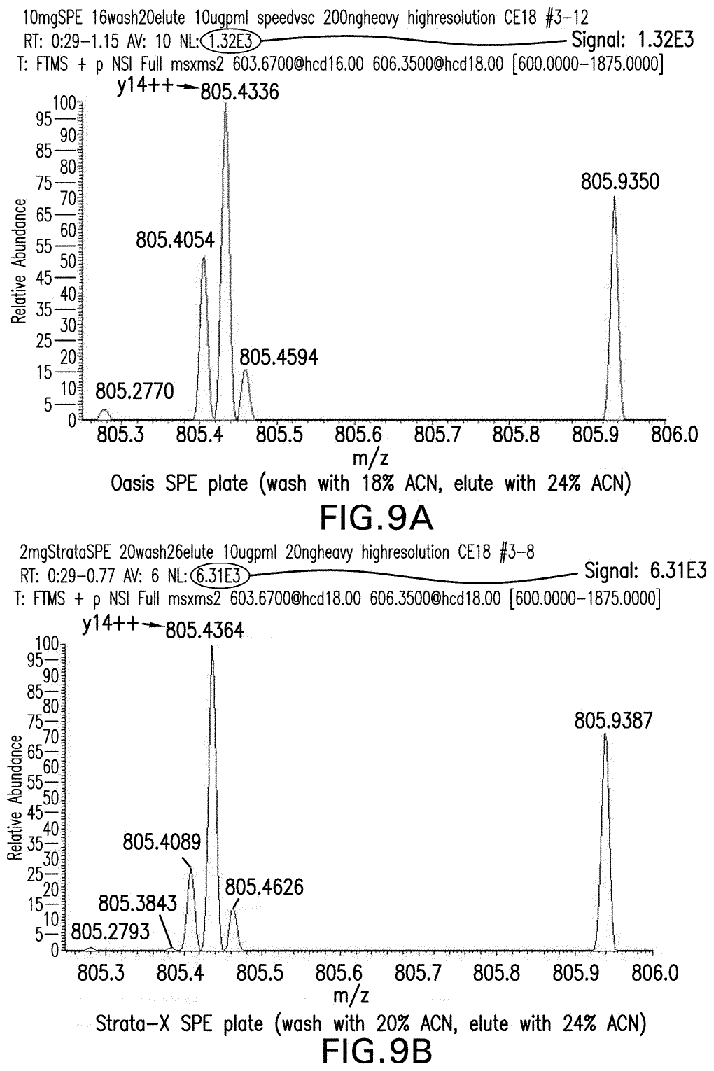

Comparison of elution profiles between Oasis HLB reverse phase 10 mg 96 plate (FIG. 9A) and Strata-X reverse phase 10 mg plate (FIG. 9B) was also performed. The data show that the 2 mg Strata-X reverse phase plate provided the strongest signal of 6.31E3 (FIG. 9B). Table 1 shows exemplary SPE parameters for fractionating the digested samples.

TABLE 1
Exemplary SPE Conditions

| | |
|---|---|
| SPE plate | 2 mg Strata-X reverse phase plate |
| Conditioning | 200 μL 0.1% formic acid in ACN |
| condition | |
| Equilibrate | 200 μL 0.1% formic acid in water |
| condition | |
| Sample | 300 μL digestion mixture at low |
| Loading | pressure (~5 psi) |
| Wash 1 | 200 μL 0.1% formic acid in 10% ACN |
| Wash 2 | 75 μL 0.1% formic acid in 20% ACN |
| Elution 1 | Add 30 μL elution solution (0.1% formic |
| | acid in 24% ACN) in the center of each |
| | well, wait for 1 min, then apply the |
| | vacuum for 5 sec. |
| Elution 2 | Add 30 μL elution solution (0.1% formic |
| | acid in 24% ACN) in the center of each |
| | well, wait for 1 min, then apply the |
| | vacuum for 60 sec. |

C. Mass Spectrometry Analysis

In one embodiment, the fractionated peptides are quantified using a mass spectrometry system containing one or more ion traps and one or more hybrid quadrupole mass filters equipped with an electrospray ionizer. An exemplary mass spectrometry system includes, but is not limited to a Thermo Q Exactive™ Plus mass spectrometer in PRM mode equipped with a TriVersa NanoMate® system for initiating nanospray ionization. This system has advanced quadrupole technology (AQT) that improves precursor selection and transmission for more-accurate quantitation of low-abundance analytes in complex matrices. The system also has sophisticated data-independent acquisition (DIA) and parallel reaction monitoring (PRM) to deliver reproducible quantitation with complete qualitative confidence. Lastly, the system has an advanced active beam guide (AABG) that reduces noise and extends maintenance intervals.

In one embodiment, quantification data is acquired using sequential PRM acquisition of endogenous and IST peptides. In some embodiments, 2-plexed PRM acquisition is used. The data for quantifying product ions are acquired in different MS² scans.

1. Internal Standard Peptide

The MS² is calibrated using a heavy isotope labeled internal standard peptide VVSVLTVLHQDWLNGK (SEQ ID NO:1). In some embodiments, the internal standard peptide is labeled with a ¹³C, ¹⁵N, and ²H, for example one or more Lys residues can be labeled with the isotope. SEQ ID NO:1 is present in all human IgG isotypes and can be reliably produced from enzyme digestion. The sequence cannot be found in any other animal species and has good MS ionization efficiency. In some embodiments the internal standard peptide is spiked into the sample to be analyzed prior to or concurrent with digestion of the proteins in the sample.

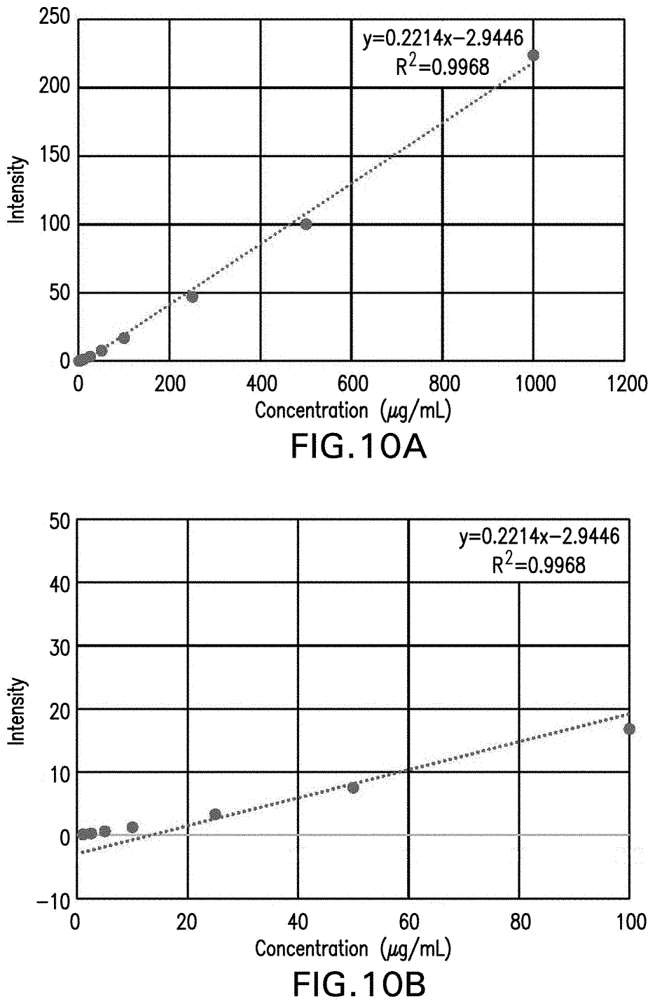

FIGS. 10A and 10B show calibration curves using SEQ ID NO:1. The HCD collision energy for MS² analysis is calibrated using a heavy isotope labeled internal standard peptide to achieve the best signal intensity for the fragment ion intended for quantitation use.

2. Internal Standard Antibody

In some embodiments, the MS² system is calibrated using an antibody labeled with a heavy isotope or a mass tag. In some embodiments, the heavy isotope is selected from the group consisting of ¹³C, ¹⁵N, and ²H. An exemplary internal standard antibody is labeled with C¹³ and N¹⁵ on one or more Lys residues. In one embodiment a SILu™MAB Stable-Isotope Labeled Universal Monoclonal Antibody Standard (human) can be used.

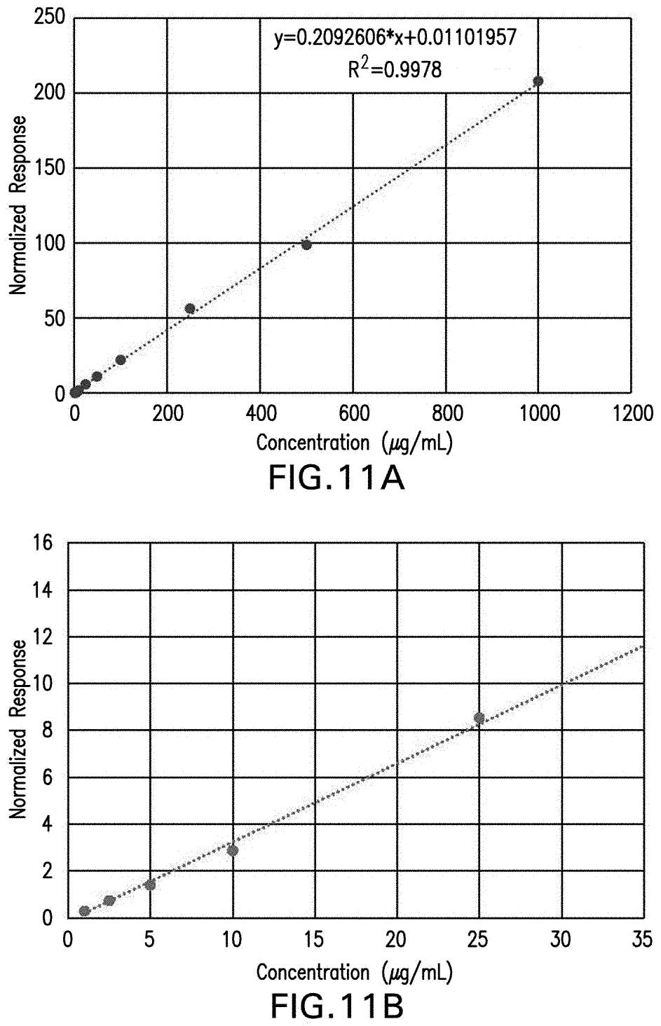

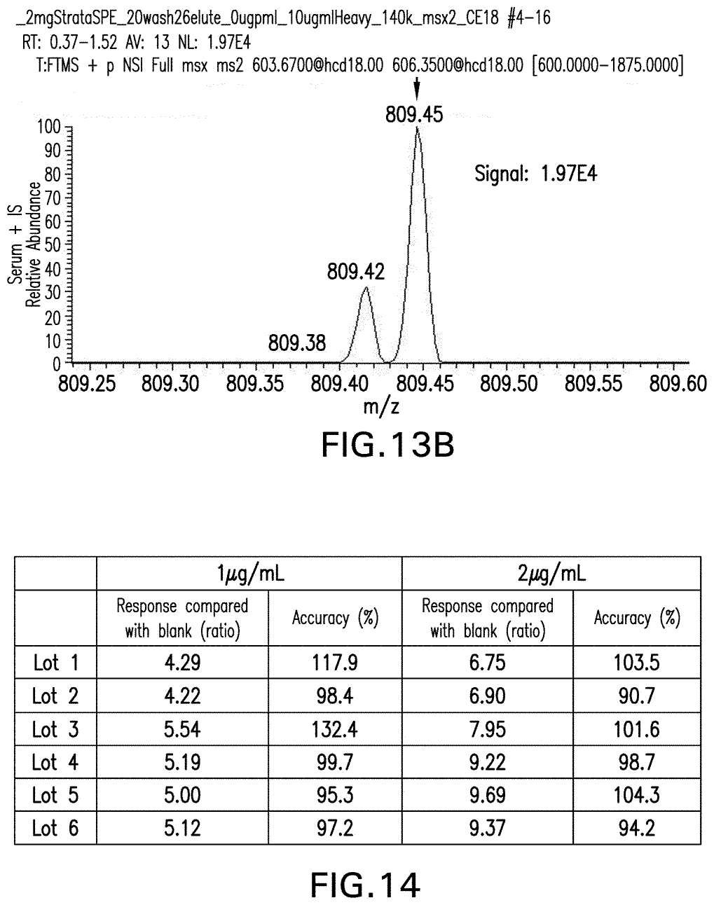

FIGS. 11A and 11B show calibration curves using the labeled internal standard antibody. FIG. 13A is a scan of a blank and 13B shows a scan with the internal standard in the blank. For FIG. 13A, one lot of monkey serum was digested by Trypsin and followed by offline SPE clean up. Then analyzed by MS using PRM method. The signal for the internal standard is very low (4.27E2). For FIG. 13B, 10 μg/mL of the internal standard was spiked into monkey serum and then digested by Trypsin and followed by offline SPE clean up. Then analyzed by MS using PRM method. As you can see the signal for the internal standard is 1.97E4. This experiment shows the blank monkey serum is free of interference for internal standard.

3. Precision and Accuracy

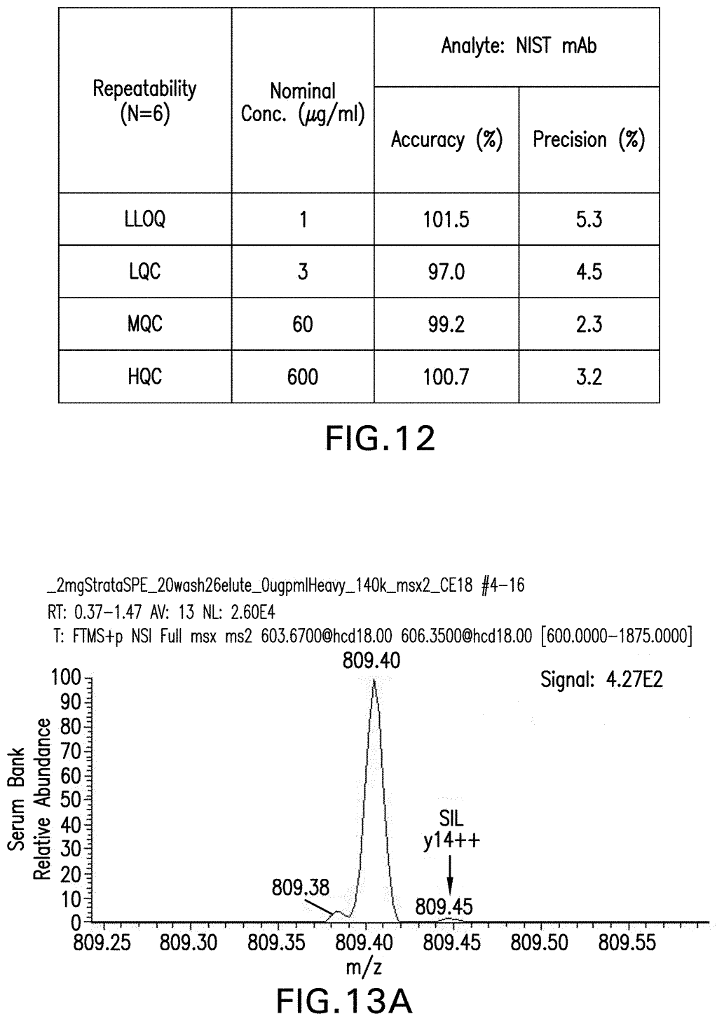

FIG. 12 describes the data obtained from quality control analysis. 4 levels of NISTmAb, Humanized IgG1k Monoclonal Antibody (Sigma-Aldrich) were spiked into monkey serum from 1 to 600 μg/mL. For each level, 6 samples were prepared independently. All samples were digested by Trypsin and cleaned up by SPE. All samples were analyzed by MS. Based on the calibration curve, the detected concentration was calculated. The accuracy was calculated by using the average detected concentration divided by nominal concentration. The precision was calculated using the % relative standard deviation (RSD) of 6 samples at each level.

4. Selectivity Analysis

FIG. 14 shows the determination of the Lower Limit of Quantification (LLOQ) using different lots of monkey blood. This experiment was performed to evaluate the matrix effect of this method. Six different lots of monkey serum were purchased, and then 1 μg/mL and 2 μg/mL NIST mAb were spiked into each lot of monkey serum separately. The signal of each lot monkey serum without NIST mAb (blank) was also detected. Then the ratio of the signal from 1 μg/mL and 2 μg/mL with the signal from the blank sample were calculated. The ratio should be at least 5 based on the requirement of method qualification from FDA. The accuracy was also calculated using the detected concentration divided by the nominal concentration. The accuracy should be within 80-120% for the LLOD based on the requirement of method qualification from the Food and Drug Administration (FDA).

5. Evaluation of Generic Applicability

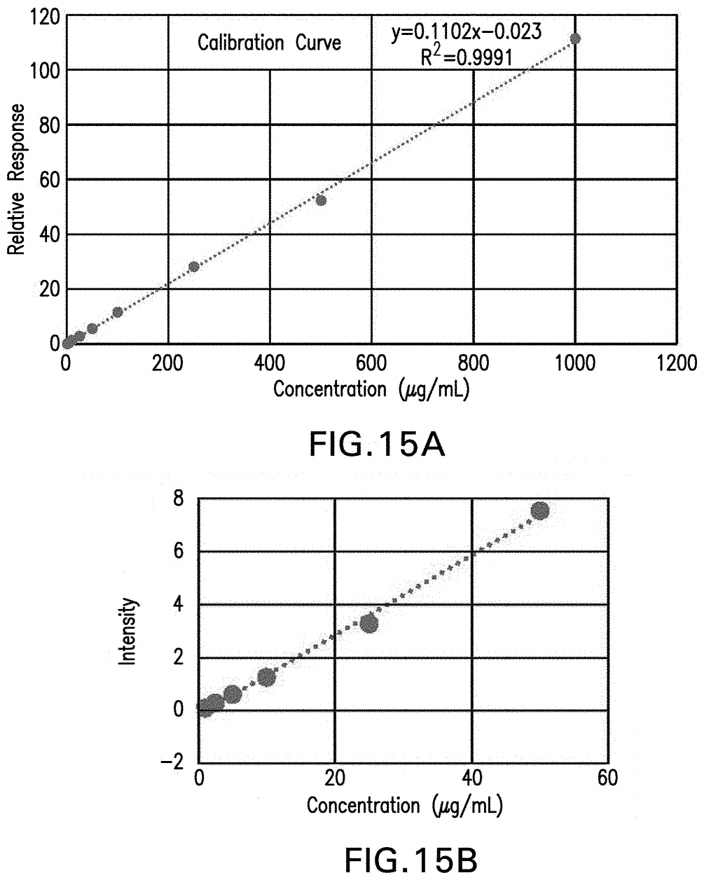

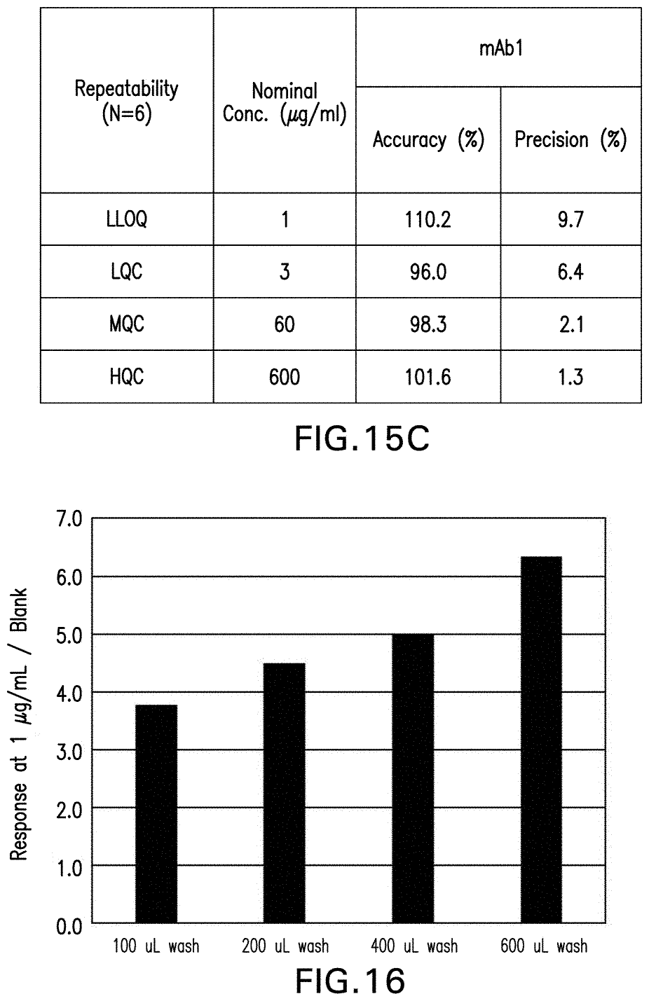

FIG. 15A shows the calibration curve generated in this method. Different concentration of NISTmAb from 1 μg/mL to 1000 μg/mL were spiked into monkey serum, and each sample was then spiked with 10 μg/mL of internal standard and followed by trypsin digestion and SPE clean up. All samples were analyzed by MS. The intensity of each sample was normalized using IS and then plotted with nominal concentration. FIG. 15B shows the zoomed region from 1 μg/mL to 50 μg/mL. As shown, the curve fits all points well in the low concentration range. FIG. 15C shows similar data as FIG. 12. The only difference is that mAb1 was used instead of NISTmAb here. mAb1 is an IgG4, and NISTmAb is a IgG1. The data show this method is suitable for both IgG1 and IgG4.

FIG. 16 is a bar graph showing that increased wash volume improves LLOQ. The X-axis represents wash volume and the Y-axis represent response at 1 μg/mL mAb/blank. The data show that increasing the wash volume during the SPE can improve the LLOD. 1 μg/mL of NISTmAb was spiked into monkey serum and the sample was digested with trypsin. During the SPE step, the plate was washed with different volumes of wash buffer while keeping the other procedure the same. When the wash volume was increase from 100 μL to 600 the ratio of the response in the sample compared with blank increased from below 4 to over 6. The ratio should be at least 5 for the LLOD based on the requirement of method qualification from FDA. So by increasing the wash volume, the LLOD was improved to 1 μg/mL.

D. Proteins of Interest

In one embodiment, the protein of interest is a protein drug product or is a protein of interest suitable for expression in prokaryotic or eukaryotic cells. For example, the protein can be an antibody or antigen-binding fragment thereof, a chimeric antibody or antigen-binding fragment thereof, an ScFv or fragment thereof, an Fc-fusion protein or fragment thereof, a growth factor or a fragment thereof, a cytokine or a fragment thereof, or an extracellular domain of a cell surface receptor or a fragment thereof. Proteins in the complexes may be simple polypeptides consisting of a single subunit, or complex multi-subunit proteins comprising two or more subunits. The protein of interest may be a biopharmaceutical product, food additive or preservative, or any protein product subject to purification and quality standards

In some embodiments, the protein of interest is an antibody, a human antibody, a humanized antibody, a chimeric antibody, a monoclonal antibody, a multispecific antibody, a bispecific antibody, an antigen binding antibody fragment, a single chain antibody, a diabody, triabody or tetrabody, a dual-specific, tetravalent immunoglobulin G-like molecule, termed dual variable domain immunoglobulin (DVD-IG), an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody. In another embodiment, the antibody comprises a chimeric hinge. In still other embodiments, the antibody comprises a chimeric Fc. In one embodiment, the antibody is a chimeric IgG2/IgG4 antibody. In one embodiment, the antibody is a chimeric IgG2/IgG1 antibody. In one embodiment, the antibody is a chimeric IgG2/IgG1/IgG4 antibody.

In some embodiments, the antibody is selected from the group consisting of an anti-Programmed Cell Death 1 antibody (e.g., an anti-PD1 antibody as described in U.S. Pat. Appln. Pub. No. US2015/0203579A1), an anti-Programmed Cell Death Ligand-1 (e.g., an anti-PD-L1 antibody as described in in U.S. Pat. Appln. Pub. No. US2015/0203580A1), an anti-DLL4 antibody, an anti-Angiopoetin-2 antibody (e.g., an anti-ANG2 antibody as described in U.S. Pat. No. 9,402,898), an anti-Angiopoetin-Like 3 antibody (e.g., an anti-AngPt13 antibody as described in U.S. Pat. No. 9,018,356), an anti-platelet derived growth factor receptor antibody (e.g., an anti-PDGFR antibody as described in U.S. Pat. No. 9,265,827), an anti-Erb3 antibody, an anti-Prolactin Receptor antibody (e.g., anti-PRLR antibody as described in U.S. Pat. No. 9,302,015), an anti-Complement 5 antibody (e.g., an anti-CS antibody as described in U.S. Pat. Appln. Pub. No US2015/0313194A1), an anti-TNF antibody, an anti-epidermal growth factor receptor antibody (e.g., an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (e.g., an anti-PCSK9 antibody as described in U.S. Pat. No. 8,062,640 or 9,540,449), an Anti-Growth and Differentiation Factor-8 antibody (e.g. an anti-GDF8 antibody, also known as anti-myostatin antibody, as described in U.S. Pat. No. 8,871,209 or 9,260,515), an anti-Glucagon Receptor (e.g. anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. Appln. Pub. No. US2014/0271681A1 or U.S. Pat. No. 8,735,095 or 8,945,559), an anti-interleukin 6 receptor antibody (e.g., an anti-IL6R antibody as described in U.S. Pat. No. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g., anti-IL33 antibody as described in U.S. Pat. No. 9,453,072 or 9,637,535), an anti-Respiratory syncytial virus antibody (e.g., anti-RSV antibody as described in U.S. Pat. No. 9,447,173), an anti-Cluster of differentiation 3 (e.g., an anti-CD3 antibody, as described in U.S. Pat. Nos. 9,447,173 and 9,447,173, and in U.S. Application No. 62/222,605), an anti-Cluster of differentiation 20 (e.g., an anti-CD20 antibody as described in U.S. Pat. No. 9,657,102 and US20150266966A1, and in U.S. Pat. No. 7,879,984), an anti-CD19 antibody, an anti-CD28 antibody, an anti-Cluster of Differentiation-48 (e.g. anti-CD48 antibody as described in U.S. Pat. No. 9,228,014), an anti-Fel d1 antibody (e.g. as described in U.S. Pat. No. 9,079,948), an anti-Middle East Respiratory Syndrome virus (e.g. an anti-MERS antibody as described in U.S. Pat. Appln. Pub. No. US2015/0337029A1), an anti-Ebola virus antibody (e.g. as described in U.S. Pat. Appln. Pub. No. US2016/0215040), an anti-Zika virus antibody, an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. Appln. Pub. No. US2016/0017029 and U.S. Pat. Nos. 8,309,088 and 9,353,176) and an anti-Protein Y antibody. In some embodiments, the bispecific antibody is selected from the group consisting of an anti-CD3×anti-CD20 bispecific antibody (as described in U.S. Pat. Appln. Pub. Nos. US2014/0088295A1 and US20150266966A1), an anti-CD3×anti-Mucin 16 bispecific antibody (e.g., an anti-CD3×anti-Muc16 bispecific antibody), and an anti-CD3×anti-Prostate-specific membrane antigen bispecific antibody (e.g., an anti-CD3×anti-PSMA bispecific antibody). In some embodiments, the protein of interest is selected from the group consisting of abciximab, adalimumab, adalimumab-atto, ado-trastuzumab, alemtuzumab, alirocumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cemiplimab, cetuximab, denosumab, dinutuximab, dupilumab, durvalumab, eculizumab, elotuzumab, emicizumab-kxwh, emtansinealirocumab, evinacumab, evolocumab, fasinumab, golimumab, guselkumab, ibritumomab tiuxetan, idarucizumab, infliximab, infliximab-abda, infliximab-dyyb, ipilimumab, ixekizumab, mepolizumab, necitumumab, nesvacumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, sarilumab, secukinumab, siltuximab, tocilizumab, tocilizumab, trastuzumab, trevogrumab, ustekinumab, and vedolizumab.

In some embodiments, the protein of interest is a recombinant protein that contains an Fc moiety and another domain (e.g., an Fc-fusion protein). In some embodiments, an Fc-fusion protein is a receptor Fc-fusion protein, which contains one or more extracellular domain(s) of a receptor coupled to an Fc moiety. In some embodiments, the Fc moiety comprises a hinge region followed by a CH2 and CH3 domain of an IgG. In some embodiments, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-1RAcP ligand binding region fused to the Il-1R1 extracellular region fused to Fc of hIgG1; see U.S. Pat. No. 6,927,004, which is herein incorporated by reference in its entirety), or a VEGF trap (e.g., aflibercept or ziv-aflibercept, which comprises the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). In other embodiments, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

### EXAMPLES

### Example 1

Materials and Methods:

Calibration standards (1, 2.5, 5, 10, 25, 50, 100, 250, 500 and 1000 μg/mL) and quality controls (QCs) (1, 3, 60 and 600 μg/mL) were prepared from the stock solutions of NISTmAb (10 mg/mL) by serial dilutions with control monkey serum. For selectivity analysis, two laboratory quality control (LQC) samples were each prepared for six different lots of blank monkey serum by spiking in NISTmAb, a humanized IgG1k monoclonal antibody, at 1 μg/mL and 2 μg/mL. 20 μL of each standard sample was spiked with 200 ng of heavy isotope labeled mAb (IS-mAb) before subjecting to trypsin digestion. Each sample was denatured, reduced and digested with trypsin for overnight followed by cleaning up using a 96 well solid phase extraction (SPE) plate. The SPE wash and elution conditions were optimized to retain the target peptide (VVSVLTVLHQDWLNGK; (SEQ ID NO:1)) and remove majority of other interferences for improved method sensitivity. Each sample was introduced to MS analysis on a Thermo Q Exactive Plus mass spectrometer in PRM mode equipped with a TriVersa NanoMate system for initiating nanospray ionization. Data was acquired using a multiplexed PRM method lasting 45 seconds for each sample.

Results:

In the search of a universal surrogate peptide for quantitation analysis, the Fc peptide VVSVLTVLHQDWLNGK (SEQ ID NO:1) was chosen because of its good MS sensitivity, presence in two human IgG subclasses (IgG1 and IgG4) commonly used in antibody therapeutics, and absence in non-human IgGs from all commonly used animal species. During the method development, the trypsin digestion conditions, SPE conditions, PRM parameters, and fragment ion choice were all optimized. The SPE condition was essential to removing most interferences while retaining majority of the surrogate peptide. The PRM parameters and fragment ion choice were key to good data accuracy and method sensitivity.

Using both NISTmAb (IgG1 subclass) and an in-house mAb4 (IgG4 subclass) as testing articles, good linearity of the calibration curve can be achieved in the tested range of 1-1000 μg/mL. The selectivity of this method was evaluated using six different lots of monkey serum, and the Lower Limit of Quantification (LLOQ) was determined to be 1-2 μg/mL in different monkey serum. In addition, the precision and accuracy of this method was tested at four different QC levels (1, 3, 60 and 600 μg/mL) with accuracy of 95%-105% and CV of <6%. Finally, with the analytical speed of <1 min per sample and zero cross-run contamination, this method can be readily applied in a high throughput environment.

Through method evaluation, this LC-free PRM-MS based method has demonstrated to be suitable for high-throughput and generic quantitation of humanized therapeutic mAbs in animal serum with a quantitation range of 2-1000 μg/mL.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof, and, accordingly, reference should be made to the appended claims, rather than to the foregoing specification, as indicating the scope of the invention.

We claim:

1. A liquid chromatography-free method for quantifying target antibodies in a sample comprising:
spiking the sample with a labeled internal standard antibody;
digesting the antibodies in the sample to produce peptides;
fractionating the peptides; and
quantifying the target antibodies using a direct infusion MS² system containing one or more ion traps and two or more quadrupole mass filters and an electrospray ionizer, wherein the method is liquid chromatography-free.

2. The method of claim 1, further comprising the step of spiking the peptides with labeled, tagged Fc peptide VVSVLTVLHQDWLNGK (SEQ ID NO:1) prior to fractionation.

3. The method of claim 1, wherein the peptides are fractionated by solid phase extraction.

4. The method of claim 3, wherein the solid phase extraction is reverse phase solid phase extraction.

5. The method of claim 1, wherein labeled internal standard antibody and the mass-tagged Fc peptide are labeled with a heavy isotope.

6. The method of claim 5, wherein the heavy isotope is selected from the group consisting of ¹³C, ¹⁵N, and ²H.

7. The method of claim 1, wherein the target antibody is a human monoclonal antibody.

8. The method of claim 1, wherein the mass spectrometry system is a tandem mass spectroscopy system.

9. A method of quantitating a protein drug product in a biological sample comprising:
spiking the sample with a known amount of a heavy mass tagged peptide standard having an amino acid sequence according to SEQ ID NO:1;
digesting protein drug product in the sample into peptides;
fractionating the peptides under conditions that retain peptides having an amino acid sequence according to SEQ ID NO:1;
analyzing the sample containing the protein drug product peptides and the peptide standards for the presence of the peptide having an amino acid sequence according to SEQ ID NO:1 using an MS² system to calibrate the system, wherein the MS² system comprises one or more ion traps and two or more quadrupole mass filters and an electrospray ionizer; and
quantitating the amount of protein drug product present in the sample based upon the presence of the peptide, wherein the method does not utilize liquid chromatography.

10. The method of claim 9, wherein the data for quantifying drug product ions and mass tagged peptide standard ions are acquired in different MS² scans.

11. The method of claim 9, wherein the peptides are fractionated using reverse phase solid phase extraction.

12. The method of claim 11, wherein the reverse phase solid phase extraction uses 15 to 25% acetonitrile as a wash and 20 to 30% acetonitrile elution.

13. The method of claim 9, further comprising spiking the sample of protein drug product with a heavy isotope-labeled protein drug product prior to digesting the sample.

14. The method of claim 9, wherein the protein drug product comprises an antibody or an antigen binding fragment thereof, a recombinant protein, a fusion protein, or a combination thereof.

15. The method of claim 1, wherein the sample comprises serum.

16. The method of claim 1, wherein the method has a dynamic range of 1 to 1000 μm/mL.

17. The method of claim 1, wherein the method has Lower Limit of Quantification (LLOQ) of 1-2 μg/mL.

18. The method of claim 1, wherein the method is an automated high throughput method.

19. The method of claim 18, wherein the method has an analytic speed of less than 1 minute per sample.


SEQUENCE LISTING (1 sequence)

SEQ ID NO: 1 - protein, 16 aa, Artificial Sequence
  Description of Artificial Sequence Synthetic peptide
VVSVLTVLHQDWLNGK